(19) United States
(12) Patent Application Publication
(10) Pub. No.: US 2007/0021654 A1
(43) Pub. Date: Jan. 25, 2007

(54) Magnetically navigable endoscopy capsule with a sensor for acquiring a physiological variable

(75) Inventors: Walter Preidel, Erlangen (DE); Johannes Reinschke, Nuremberg (DE)

Correspondence Address: STAAS & HALSEY LLP, SUITE 700, 1201 NEW YORK AVENUE, N.W., WASHINGTON, DC, 20005, US

(73) Assignee: SIEMENS AKTIENGESELLSCHAFT, Munich (DE)

(21) Appl. No.: 11/482,932

(22) Filed: Jul. 10, 2006

(30) Foreign Application Priority Data
Jul. 8, 2005 (DE) 10 2005 032 378.2

(51) Int. Cl. A61B 1/06; A61B 1/00

(52) U.S. Cl. 600/160; 600/114; 600/130

(57) ABSTRACT

A permanent magnetic element is included in an endoscopy capsule that is magnetically navigable in a hollow organ. At least one physiological sensor, such as a conductivity sensor, is provided in a subregion of the surface. The physiological sensor may be have a lateral cylindrical surface and transmit a signal to the outside. The capsule may be set rotating by magnetic forces to enable 360 degrees of detection. Alternatively, the sensor can extend around the entire periphery of the capsule surface.

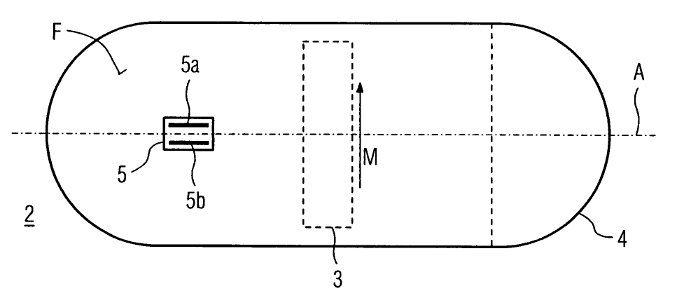

### CROSS REFERENCE TO RELATED APPLICATIONS

This application is based on and hereby claims priority to German Application No. 2005 032 378.2 filed on Jul. 8, 2005, the contents of which are hereby incorporated by reference.

### BACKGROUND OF THE INVENTION

1. Field of the Invention

The invention relates to an endoscopy capsule.

2. Description of the Related Art

Minimal or noninvasive medical techniques are used to examine or treat a human or animal as test subject. The use of endoscopes that are introduced into the test subject through body openings or small incisions has been known for quite some time. In this case, inspection or manipulation units for example a camera or a gripper, are located on the tip of a flexible basic body of greater or lesser length in order to execute the desired activity. Because of frictional effects and the limited length and flexibility of endoscopes, the latter can be used only in an appropriate restrictive fashion.

DE 101 42 253 C1 discloses in relation to endoscopy, an apparatus and a method that operate in a wireless fashion. To this end, a so called “endorobot” in the form of an (endoscopy) capsule of approximately 2 cm length and approximately 1 cm diameter includes an inspection or diagnosis or therapy device. Such devices can be, for example, a video camera, a biopsy forceps, a clip or a drug reservoir. Moreover, it is proposed for the known capsule to install probes with sensors for physiological variables such as temperature, electric conductivity, pH value, pressure or, if appropriate, also chemical sensors. However, no details are mentioned as to how a corresponding sensor is to be fashioned.

A known endoscopy capsule further includes a magnetizable or permanently magnetic element. The capsule is moved and/or navigated in the test subject in a wireless fashion. To this end, the test subject lies entirely or partially in a solenoid system composed of a number of, for example, 14 individually excitable coils (compare DE 103 40 925 A1). The coil system generates suitable magnetic fields or gradient magnetic fields which, via its magnetic element, generate at the capsule located in the test subject forces and/or torques in order to be able to propel or rotate the capsule in the test subject. It is possible in this way for the capsule to navigate in the test subjects without making contact. The fields of use of appropriate endoscopy apparatuses are here, chiefly, hollow organs, in particular the human gastrointestinal tract, which can be traversed in its entirety with the aid of the capsule in a single pass.

Furthermore, it has already been known for some time from non-navigable endoscopy capsules to acquire physiological variables at the inner wall of tubular hollow organs such as the gastrointestinal tract of a test subject. Thus, for example, the capsule with radio location, which is to be gathered from US 2003/019814 A1, can be fashioned in a way known per se as a video capsule in order to identify an acute hemorrhage in the small intestine, for example. However, if the hemorrhage to be identified is small and closely restricted locally, it can be overlooked by the video capsule.

US 2002/0132226 further discloses an endoscopy capsule that is to be ingested orally, or swallowed by a test subject. This capsule is provided on its outer surface with a sensor membrane with the aid of which electrochemically detectable variables (so called “biosensing”) such as the pH value or specific enzymes are to be acquired. The sensor signals obtained are then further processed in the capsule and transmitted by radio to a receiving unit arranged outside the test subject.

It is to be assumed with these known endoscopy capsules that the sensors installed in these capsules only have a locally closely restricted measuring range for physiological parameters as is the case, for example, for known video capsules whose camera, which is directed forward or backward, has an angular aperture of at most 140°. Otherwise, there is a need for complicated additional measures as in the case of the endoscopy capsule to be gathered from U.S. Pat. No. 4,217,045 A, which enables a panoramic detection. In the case of the known capsule, a photographic unit is located for this purpose inside an inflatable balloon which can be illuminated and through which detection is carried out via various optical devices such as lenses and mirrors.

### SUMMARY OF THE INVENTION

It is an aspect of the present invention to fashion the endoscopy capsule of the type mentioned at the beginning so as to enable physical variables to be detected in a panoramic fashion in conjunction with magnetic navigation.

A first achievement of this object is accomplished with an endoscopy capsule having
an elongated shape with a surface part of its surface that surrounds its longitudinal axis and is at least approximately in the shape of a lateral cylinder surface,
in the interior, a permanent magnetic element with a magnetization perpendicular to the longitudinal axis,
the ability to be navigated by magnetic forces, acting on the permanent magnetic element, in a hollow organ of a test subject that surrounds the capsule, such as in a gastrointestinal tract,
at least one sensor for acquiring a physiological variable inside the hollow organ, and
a way to transmit the sensor signals to a receiving unit arranged outside the test subject.

Preferably, the at least one sensor is arranged on the surface part of the capsule surface, the capsule can be rotated about its longitudinal axis such that the at least one sensor is capable (in practice) of detecting the region of the hollow organ extending around the entire circular periphery of the capsule surface. Rotation is preferably to be executed by an external solenoid system surrounding the test subject with the capsule.

The capsule need not necessarily have a surface part with the sensor that is exactly in the shape of a lateral cylinder surface. Since this surface part need only be at least approximately in the shape of a lateral cylinder surface, shapes that deviate from the exact shape of a lateral cylinder surface and are rotationally symmetrical with reference to the longitudinal axis of the capsule such as, for example, a shape of a paraboloid of rotation are also to be included.

A further achievement of the object is accomplished in the case of an endoscopy capsule having the features named at the beginning, its at least one sensor can occupy the entire circular periphery of the capsule surface at least largely (except for minor interruptions by the line of action of the sensor).

Of course, it is also possible for the two approaches to achievements set forth above to be combined with one another.

The advantages achieved with the aid of the inventive measures are to be seen, in particular, in reduced times of examination and/or treatment by the examiner such as, for example, a doctor in conjunction with the same results, or better ones, because—by contrast with a movement of a catheter tip of a classic endoscope—the movement of the capsule can be programmed and thereby largely automated. A 360° detection range is to be ensured, in particular, such that even locally closely restricted sites of a hollow organ that is to be examined can be diagnosed and/or treated.

Advantageous refinements of the endoscopy capsule may include the following features:
The at least one sensor can be designed as a conductivity sensor for determining the electric conductivity. In this case, the starting point is the fact that many diagnostic statements in medicine are to be obtained by measuring the conductivity of bodily fluids or juices.
In this case, the at least one conductivity sensor can advantageously be designed in the form of a resistance bridge circuit. Specifically, such resistance bridges can be used to identify overshooting of a defined limiting value of the conductivity in a simple way.
It is preferred to apply to the at least one conductivity sensor an AC voltage whose frequency is, in particular, at least 100 Hz, preferably at least 1 kHz or more.
The conductivity sensor can be designed with particular advantage for detecting blood.
Of course, in addition to the at least one sensor for acquiring a physiological variable at least one further sensor system such as, for example, a video camera and/or a temperature sensor and/or a pH sensor and/or a chemical sensor can also be present. The signal evaluation and transmission can then advantageously be combined with that of the physiological sensor system.
A wireless navigation of the endoscopy capsule can advantageously be provided. No wire connections of any sort to the capsule exist in this case. Such a wireless navigation comes into consideration, in particular, whenever the aim is for the capsule to be navigated magnetically without making contact, since then the freedom of movement of the capsule is not impaired by any sort of mechanical connection, in particular to a catheter.
Instead of this, however, it is also possible to provide a connection by a minicatheter, the latter lacking a shear strength adequate for mechanical capsule navigation. In this case, such a minicatheter can advantageously be used to create a signal connection, and/or energy connection, and/or bonded connection such as, for example, for the purpose of feeding chemical substances or a rinsing liquid.
The navigation of the capsule can be executed using a known external solenoid system surrounding the test subject with the capsule in the case of the embodiment of an endoscopy capsule.

### BRIEF DESCRIPTION OF THE DRAWINGS

These and other objects and advantages of the present invention will become more apparent and more readily appreciated from the following description of the preferred embodiments, taken in conjunction with the accompanying drawing of which:

The figure is a schematic longitudinal section of a preferred exemplary embodiment of an endoscopy capsule according to the invention.

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

Capsules known per se (compare, for example, DE 101 42 253 C1 mentioned at the beginning, or US 2002/0132226 A1 mentioned) are the starting point in the case of the embodiment of the endoscopy capsule, designated in general by 2, illustrated in the figure. The capsule 2 is configured with an elongated shape and has a surface part F of its outer surface that is in the shape of a lateral cylinder surface and concentrically surrounds its longitudinal axis A. Located inside the capsule as a magnetic element is a rod-shaped permanent magnet 3 whose magnetization M is aligned perpendicular to the longitudinal axis A of the capsule. It is intended to be possible to insert the capsule into a hollow organ of a test subject such as, for example, the gastrointestinal tract of a human being, and to be possible to navigate it there in a wireless fashion, that is to say without a mechanical and/or electrically conducting connection to the outside. This purpose is served by a solenoid system known per se (compare, for example, DE 103 40 925 A1), with the aid of which settable magnetic fields and gradient magnetic fields can be generated, and the permanent magnet 3 and thus the capsule 2 rigidly connected to it can be moved in a prescribed way. That is to say, suitably controlled currents through the individual solenoids or the solenoid system can be used to set the capsule 2 in a screwing movement, that is to say simultaneously rotating about its longitudinal axis A and being displaced along the longitudinal axis, or alternately executing a rotational movement or a stroke movement, in which case a 360° rotation about the longitudinal axis is followed by a displacement along this axis. In this case, the alternating rotational/stroke movement is to be preferred from the point of view of a restricted power consumption by the solenoid system.

Instead of such a completely wireless navigation, it is, if appropriate, also possible to equip the capsule with a highly flexible minicatheter, or to connect it to one. Such a minicatheter can be provided, for example, for examinations of the large intestine in the case of which the magnetically navigable capsule is inserted rectally. It is true that such a minicatheter lacks adequate shear strength for a mechanical capsule navigation, but it does enable supply and/or removal of electric energy and, if appropriate, of substances such as, for example, a rinsing liquid, which because of the relatively small overall size of the capsule cannot be stored in sufficient quantity therein or cannot be transported.

The capsule 2 is, furthermore, expediently equipped with a video camera known per se of which only its camera window 4 is to be seen in the figure. The video signal obtained with the camera is further processed in the capsule and transmitted by radio to an external receiving unit, that is to say one located outside the test subject. Both the positional (3D) and, if appropriate, a rotational angle signal (rotary position signal) (2D or 3D) are also transmitted and received in this way. The rotational angle about the capsule longitudinal axis need not necessarily be measured in this case, but is yielded from the alignment of the basic magnetic field of the magnet system in the plane perpendicular to the capsule longitudinal axis A. Specifically, in this plane the permanent magnet 3 in the capsule 2 is aligned along the outer basic field. That is to say, instead of a 6D positional measurement (with reference to 3 centroid coordinates and 3 solid angles of the capsule orientation) it is also possible, in the manner described, to provide a 5D positional measurement in which no measurement is made of the rotational angle about the capsule in the longitudinal axis.

At least one sensor for at least one physiological variable is fitted at or on the cylindrical surface part F of the capsule surface. The electric conductivity may be selected below as preferred physiological variable for the exemplary embodiment. Like the video and/or positional signal, the measured physiological value is transmitted by radio to the or another external receiving unit, and further processed there.

It is possible in this way to scan the entire inner wall of the hollow organ, such as the gastrointestinal tract, to be examined. The conductivity measurement can be implemented for different requirement profiles with a different need for space and energy.

The measurement of the electric conductivity can be performed as follows: The simplest measuring method is detuning of a resistance bridge that, for example, in the case of undershooting of the resistance between electrodes, that is to say overshooting of a defined limiting value of the conductivity, supplies the intestinal juice with a digital one-bit signal with the following content: “setpoint of conductivity overshot”. The telemetric transmission is therefore particularly simple. If required, the measurement can be performed to a number of places of accuracy.

It is basically advantageous to measure the conductivity with AC voltage whose frequency is, for example, at least 100 Hz or more, preferably 1 kHz or more. Electrode polarization and electrolyte decomposition can largely be minimized or even excluded in this way. Platinum can preferably be used as electrode material. Specifically, the biocompatibility and functional stability of platinum as electrode material is known. Two platinum electrodes of the sensor 5 are indicated in the figure and denoted by 5a and 5b, respectively.

The design as an appropriate blood sensor for examination in the gastrointestinal tract of a patient may be indicated below:

At least one conductivity sensor 5 with platinum electrodes 5a and 5b is integrated on the surface F, in the shape of a lateral cylinder surface, of a known endoscopic video capsule 2. Alternatively, the patient swallows two capsules: one video capsule and one conductivity capsule 2, the two capsules being magnetically navigable and being equipped with radio location and transmission. The capsule 2 with the conductivity sensor can execute a combined rotary/thrust movement by external magnetic navigation. An increased conductivity is identified at the site of an acute hemorrhage, and at the same time a typical red coloration by the video signal is visible. By correlating the two measurement signals with reference to video and conductivity via position, a more reliable hemorrhage indicator is then obtained than is the case solely with the video signal. Such a positionally referred correlation analysis between measured value series of a number of sensors with the aid of position measuring signals is also required when the at least 2 sensors, for example a camera in addition to the conductivity sensor, are arranged jointly in a capsule since, as is indicated in the figure, two sensors cannot be fitted at the same place in or on the capsule. The means and methods to be provided for such a correlation analysis are generally known.

As an alternative to the scanning function, assumed above, by rotary movement of the capsule about its longitudinal axis, the at least one sensor can also be designed as a 360° sensor; that is to say, this sensor extends over the entire periphery of the capsule or its surface part in the shape of a lateral cylinder surface. In this case, it is possible if appropriate to dispense with a rotary movement of the capsule. However, sensor outlay is higher.

Furthermore, the at least one sensor of an inventive endoscopy capsule also need not necessarily be a conductivity sensor, although is preferred to provide such a one. Thus, the sensor can also be designed for acquiring other physiological (including physical) values, such as the temperature, the pH value or occurrence/concentration of substances typical of specific illnesses.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 358 F3d 870, 69 USPQ2d 1865 (Fed. Cir. 2004).

What is claimed is:

1. An endoscopy capsule for use in a hollow organ of a test subject that surrounds the capsule when in use and with a receiving unit outside the test subject, comprising:
a surface, elongated and surrounding a longitudinal axis, substantially forming a lateral cylinder with an interior;
a permanently magnetic element in the interior, with a magnetization perpendicular to the longitudinal axis, affixed to the surface and causing the endoscopy capsule to move in response to magnetic forces acting on the permanently magnetic element;
at least one sensor, arranged on the elongated surface of the endoscopy capsule, acquiring a physiological variable of a region inside the hollow organ extending around the entire circular periphery of the capsule surface; and
means for transmitting sensor signals from the at least one sensor to the receiving unit arranged outside the test subject.

2. The endoscopy capsule as recited in claim 1, wherein the hollow organ is a gastrointestinal tract.

3. The endoscopy capsule as claimed in claim 1, wherein the magnetic forces acting on the permanently magnetic element are generated by an external solenoid system surrounding the test subject when the endoscopy capsule is used.

4. The endoscopy capsule as claimed in claim 3, wherein the at least one sensor is a conductivity sensor detecting electric conductivity.

5. The endoscopy capsule as claimed in claim 4, wherein the at least one conductivity sensor is a resistance bridge circuit.

6. The endoscopy capsule as claimed in claim 5, wherein the at least one conductivity sensor receives an alternating current voltage.

7. The endoscopy capsule as claimed in claim 6, wherein the alternating current voltage has a frequency of at least 100 Hz.

8. The endoscopy capsule as claimed in claim 7, wherein the alternating current voltage has a frequency of at least 1 kHz.

9. The endoscopy capsule as claimed in claim 7, wherein the at least one conductivity sensor has at least one platinum electrode.

10. The endoscopy capsule as claimed in claim 9, wherein the at least one conductivity sensor detects blood.

11. The endoscopy capsule as claimed in claim 10, further comprising a further sensor system is also present in addition to the at least one sensor.

12. The endoscopy capsule as claimed in claim 11, wherein a further sensor system includes at least one of a video camera, a temperature sensor, a pH sensor and a chemical sensor.

13. The endoscopy capsule as claimed in claim 12, further comprising means for a positional correlation analysis between measured value series is assigned to the at least one sensor and the further sensor system.

14. The endoscopy capsule as claimed in claim 13, wherein the endoscopy capsule is driven by wireless navigation.

15. The endoscopy capsule as claimed in claim 13, further comprising a connection to a minicatheter.

16. The endoscopy capsule as claimed in claim 15, wherein the minicatheter produces at least one of a signal connection, an energy connection, and a bonded connection.

17. An endoscopy capsule for use in a hollow organ of a test subject that surrounds the capsule when in use and with a receiving unit outside the test subject, comprising:
an elongated surface having an interior, surrounding a longitudinal axis and substantially forming a lateral cylindrical surface,
a permanent magnetic element in the interior, with a magnetization perpendicular to the longitudinal axis, providing navigation of the endoscopy capsule in the hollow organ of the test subject by magnetic forces acting on the permanently magnetic element;
at least one sensor, substantially surrounding an entire circular periphery of the elongated surface, acquiring a physiological variable inside the hollow organ; and
means for transmitting the sensor signals to the receiving unit outside the test subject.

18. The endoscopy capsule as recited in claim 17, wherein the hollow organ is a gastrointestinal tract.

19. The endoscopy capsule as claimed in claim 17, wherein the magnetic forces acting on the permanently magnetic element are generated by an external solenoid system surrounding the test subject when the endoscopy capsule is used.

20. The endoscopy capsule as claimed in claim 19, wherein the at least one sensor is a conductivity sensor detecting electric conductivity.

21. The endoscopy capsule as claimed in claim 20, wherein the at least one conductivity sensor is a resistance bridge circuit.

22. The endoscopy capsule as claimed in claim 21, wherein the at least one conductivity sensor receives an alternating current voltage.

23. The endoscopy capsule as claimed in claim 22, wherein the alternating current voltage has a frequency of at least 100 Hz.

24. The endoscopy capsule as claimed in claim 23, wherein the alternating current voltage has a frequency of at least 1 kHz.

25. The endoscopy capsule as claimed in claim 23, wherein the at least one conductivity sensor has at least one platinum electrode.

26. The endoscopy capsule as claimed in claim 25, wherein the at least one conductivity sensor detects blood.

27. The endoscopy capsule as claimed in claim 26, further comprising a further sensor system is also present in addition to the at least one sensor.

28. The endoscopy capsule as claimed in claim 27, wherein a further sensor system includes at least one of a video camera, a temperature sensor, a pH sensor and a chemical sensor.

29. The endoscopy capsule as claimed in claim 28, further comprising means for a positional correlation analysis between measured value series is assigned to the at least one sensor and the further sensor system.

30. The endoscopy capsule as claimed in claim 29, wherein the endoscopy capsule is driven by wireless navigation.

31. The endoscopy capsule as claimed in claim 29, further comprising a connection to a minicatheter.

32. The endoscopy capsule as claimed in claim 31, wherein the minicatheter produces at least one of a signal connection, an energy connection, and a bonded connection.
